(19) United States
(12) Patent Application Publication
(10) Pub. No.: US 2011/0166439 A1
(43) Pub. Date: Jul. 7, 2011

(54) MEDICAL INSTRUMENT

(75) Inventors: Joachim Pfeffer, Aachen (DE); Klaus Düring, Frechen (DE)

(73) Assignee: MaRVis Technologies GmbH, Frechen (DE)

(21) Appl. No.: 12/993,428

(22) PCT Filed: May 25, 2009
(86) PCT No.: PCT/EP2009/003685

§ 371 Date: Feb. 10, 2011

(30) Foreign Application Priority Data
May 23, 2008 (DE) 10 2008 024 976.9

(51) Int. Cl. A61B 5/055; A61B 6/00

(52) U.S. Cl. 600/411

(57) ABSTRACT

The invention relates to a medical instrument that can be inserted into a human or animal body. The medical instrument includes an instrument body having at least one poor electrically conducting rod-type body made of a matrix material and non-metallic filaments. The medical instrument is characterized in that the rod-shaped body is doped with X-ray marker particles, and the medical instrument includes an MR-marker, or the instrument body includes an immobilized active MR marker in the surface area.

The present invention relates to a medical instrument. In particular, the present invention concerns a medical instrument which can be detected by means of magnetic resonance tomography.

WO 2007/000148 A2 discloses a rod-type body serving for forming medical instruments such as catheters or guiding wires for catheters. This rod-type body consists of one or more filaments and a non-ferromagnetic matrix material enclosing the filaments. A doping agent made of particles which create MRT artifacts is introduced into the matrix material.

A detailed explanation of magnetic resonance tomography (MRT) or magnetic resonance imaging can be found in the Internet at http:/en.wikipedia.org/wiki/MRT.

U.S. 2003/0055449 A1 shows a balloon catheter in which the balloon is formed from a polymeric material comprising a ferromagnetic or paramagnetic material so that it is visible during the magnetic resonance examination.

U.S. Pat. No. 5,154,179 discloses a catheter which is formed e.g. from an extruded plastic hose, ferromagnetic particles being contained in the plastic material of the plastic hose. This catheter is visible in magnetic resonance tomography. Further, it is suggested to provide such a catheter with a material which is opaque for X-rays. It is preferred to use non-ferrous materials for these X-ray markers.

DE 101 07 750 A1 describes a guiding wire which is supposed to be suitable for magnetic resonance tomography. This guiding wire comprises a core made of a metallic front part. Ropes made of an electrically non-conductive plastic material are arranged between an outer jacket and the core. This plastic material is supposed to be reinforced with glass fibers or carbon fibers. Carbon fibers are, however, electrical conductors so that they cannot be used for magnetic resonance tomography.

Further, medical equipment is known from EP 1 206 945 A1, which is provided with paramagnetic metallic compounds and/or a paramagnetic metal so that they are visible in a magnetic resonance imaging process.

WO 87/02893 discloses poly-chelating substances for the imaging enhancement and spectral enhancement for magnetic resonance imaging. These substances comprise different complexes in which metal ions, in particular gadolinium ions are immobilized.

The relaxivity of gadolinium(III) complexes is explained in chapter 1.6.1 of the inaugural dissertation by Daniel Storch, entitled “Neue, radioaktiv markierte Magnet-Resonanz-aktive Somatostatinanaloga zur besseren Diagnose and zielgerichteten Radionuklid-therapie von neuroendokrinen Tumoren”, Basel, 2005. The paramagnetic relaxation of the water molecules which are in the vicinity of the gadolinium(III) ion is the result of the dipole-dipole-interaction between the nuclear spin and the fluctuating local magnetic field of the magnetic resonance imaging apparatus, caused by the unpaired electrons. The magnetic field around the paramagnetic center, i.e. the gadolinium(III) ion, disappears with increasing distance. This is why it is decisive to bring the protons in close proximity to the metal ion. Concerning gadolinium(III) complexes, this means that the water molecules are to be transported into the first coordination sphere of the metal ion. These “inner-sphere” H₂O molecules are exchanged with the surrounding water molecules and transmit the paramagnetic effect in this way.

DE 100 40 381 C1 discloses fluoroalkyl-containing complexes with residual sugars. These complexes can be provided with paramagnetic metal ions so that they can serve as contrast agents in magnetic resonance imaging. These metal ions are in particular the bivalent and trivalent ions of the elements of the atomic numbers 21 to 29, 42, 44 and 58 to 70. Suitable ions are, for instance, the chromium(III), iron(II), cobalt(II), nickel(II), copper(II), praseodymium(III), neodymium(III), samarium(III) and ytterbium(III) ions. Gadolinium(III), erbium(III), dysprosium(III), holmium(III), erbium(III), iron(III) and manganese(II) ions are particularly preferred because of their strong magnetic moment.

EP 1 818 054 A1 discloses the use of gadolinium chelates for the purpose of marking cells.

U.S. Pat. No. 6,458,088 B1 describes a guiding wire provided for magnetic resonance imaging, this guiding wire comprising a glass body. The glass body is provided with a protective layer which is made of polymeric material and can be additionally provided with fibers. The distal end of the guiding wire can be formed from a metal section such as nitinol. This metal section should have a length which is clearly shorter than the wavelength of the magnetic resonance field.

WO 2005/120598 A1 discloses a catheter guiding wire comprising a PEEK core. This core is provided with a coating. The coating is provided with a contrast agent. The contrast agent is iron powder having a grain size of less than 10 μm.

WO 97/17622 discloses a medical instrument comprising an electrically non-conductive body which is provided with an ultra-thin coating made of an electrically conductive material so that the medical instrument is visible in a magnetic resonance tomography process without unduly affecting the image.

WO 99/060920 A and WO 2002/022186 A each show a coating for a medical instrument comprising a paramagnetic ion which is complexed in the coating. The paramagnetic ion is in particular gadolinium. This coating is visible during the MRT examination.

The invention is based on the object to provide a medical instrument which can be inserted in a human or animal body and is very versatile as regards its use in an MRT examination.

This object is achieved by a medical instrument comprising the features of claim 1 or 2. Advantageous designs are indicated in the sub-claims.

According to a first aspect of the present invention, a medical instrument is provided which can be inserted in a human or animal body, the medical instrument including an instrument body. The instrument body comprises at least one rod-type body having poor electrical conductivity and being formed from a matrix material and non-metallic filaments. This medical instrument is distinguished in that the rod-type body is doped with an X-ray marker and the medical instrument comprises an MR marker.

By providing an X-ray marker as well as an MR marker, the medical instrument can be seen in both X-ray examinations and MRT. The introduction of the X-ray marker into the medical instrument can be easily realized by the use of a rod-type body having an appropriate doping. Such rod-type bodies can be produced as a mass product with different doping agents at a favorable price and with an exact dosage of the marker particles. During the production of a medical instrument, the visualization of the medical instrument in X-ray examinations can be ensured by using the respective rod-type body with an X-ray marker.

According to a second aspect, the medical instrument according to the invention is designed for being inserted into a human or animal body, said instrument comprising an instrument body having a surface which may come into contact with the human or animal body. The surface area of the instrument body is provided with immobilized active MR markers.

Active MR markers are markers which interact with the protons in the water or fat molecule and result in a quicker relaxation of the protons adjoining the marker when these have undergone an induced orientation due to the applied magnetic field. The reduction of the relaxation time caused by the marking process results in strong MRT signals, bringing about a correspondingly high contrast in the images created hereby.

By the use of an immobilized active MR marker on the surface of the instrument body in connection with at least one rod-type body doped with a marker, the high contrast of an active MR marker in MRT and the versatile field of application of passive markers is combined in a simple way. The passive markers may be designed both for X-ray and MRT examinations. It is preferred that the medical instrument comprises several rod-type bodies which are doped differently.

Medical instruments provided with active MR markers on their surface have a very flexible field of application with respect to the sequences used in an MRT examination and also are uniformly visible in MRT examinations with different sequences.

The active MR markers comprise an element or a combination of elements or a compound of an element from the group consisting of gadolinium, cerium, praseo-dymium, neodymium, promethium, samarium, europium, terbium, dysprosium, holmium, erbium, thulium, ytterbium and lutetium. These elements can be bound in a complex in the form of ions. They can also be present, however, in the form of salts or alloys.

It is particularly preferred that gadolinium is used as an active MR marker. This element is preferably immobilized by means of a complex, in particular a chelate complex.

The complexes can either be covalently bound to the surface of the instrument body or embedded in a coating which is capable of swelling and formed on the surface of the instrument body.

Spacers can be arranged between the complexes and the surface of the instrument body so that the active MR marker is arranged so as to be spaced from the surface of the instrument body. This measure makes sure that the body fluid flows over and around the markers and the majority of the MR markers is in close proximity to protons of water and/or fat molecules.

When a coating is provided which is capable of swelling and contains the MR markers, body fluid is absorbed by the coating capable of swelling while the medical instrument is inserted in the human or animal body so that protons of water molecules will bind closely to the MR markers, resulting in the interaction which shortens the relaxation time.

The invention will now be exemplified in more detail on the basis of the embodiments illustrated in the drawings in which:

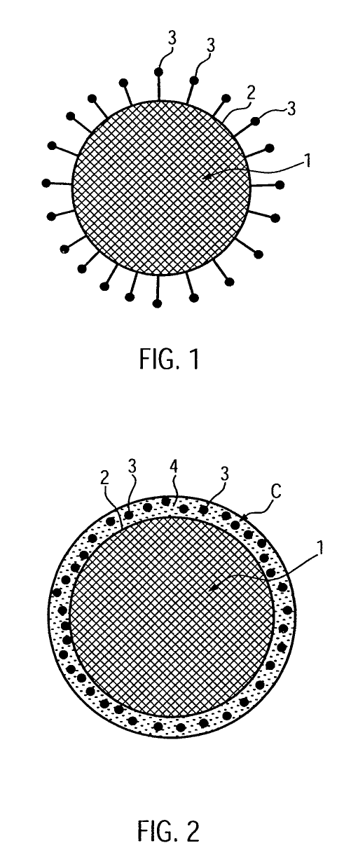

FIG. 1 shows a guiding wire according to a first embodiment of the invention in cross-section,

FIG. 2 shows a guiding wire according to a further embodiment of the present invention in cross-section,

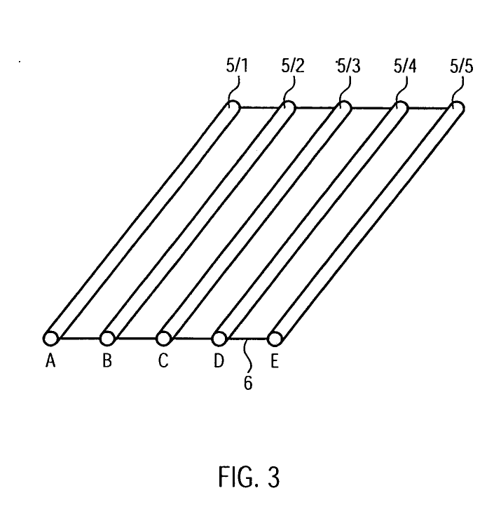

FIG. 3 shows a test equipment with several rods which are provided with different markers,

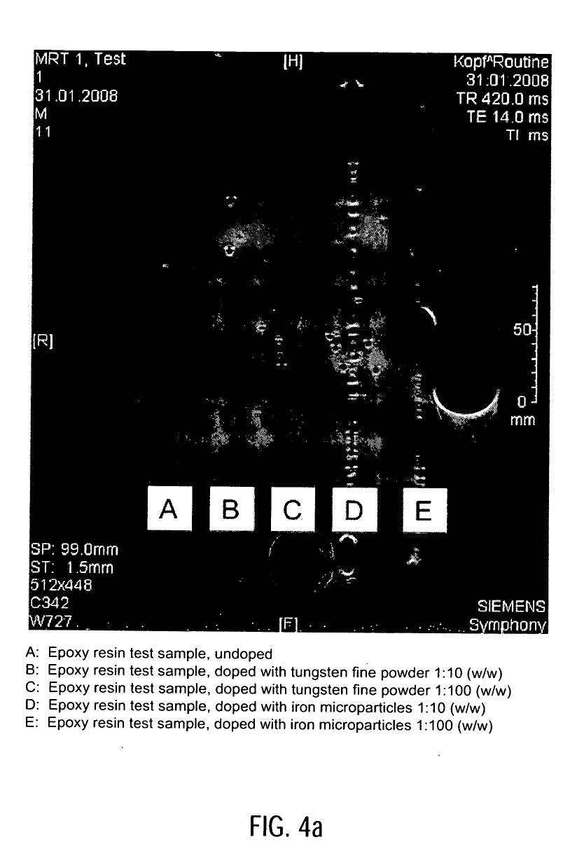

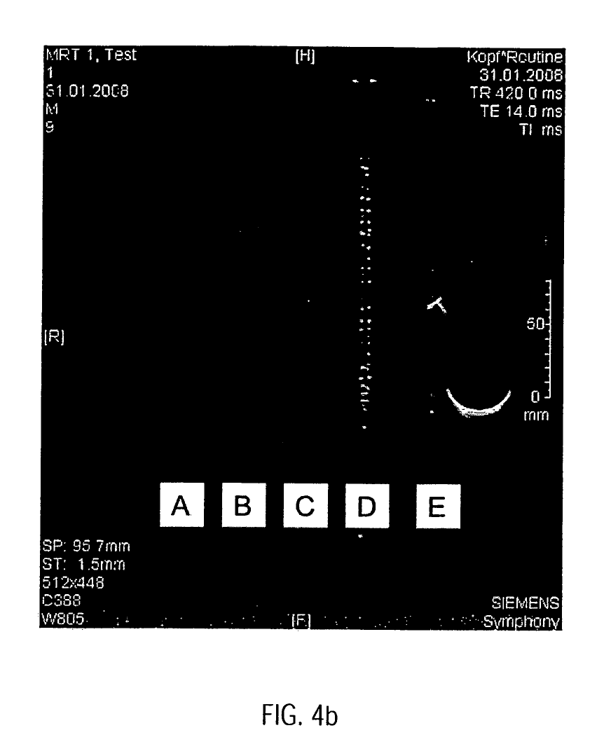

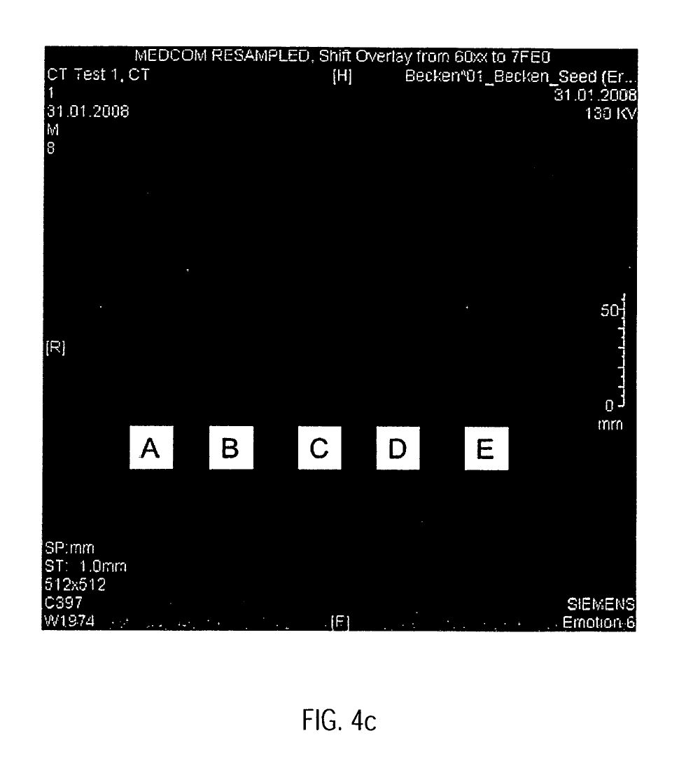

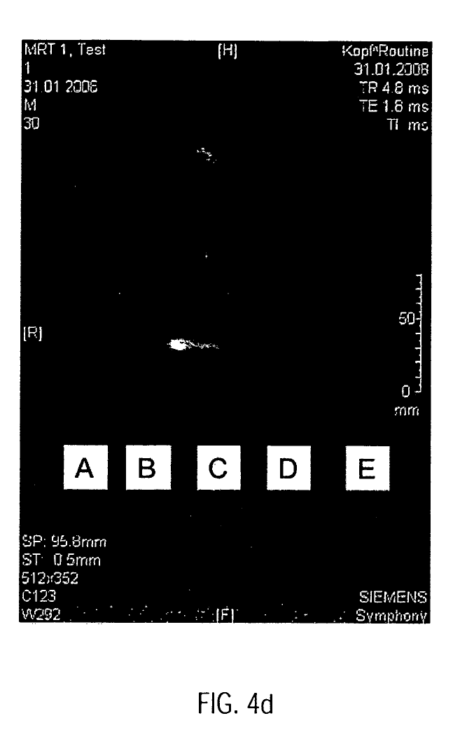

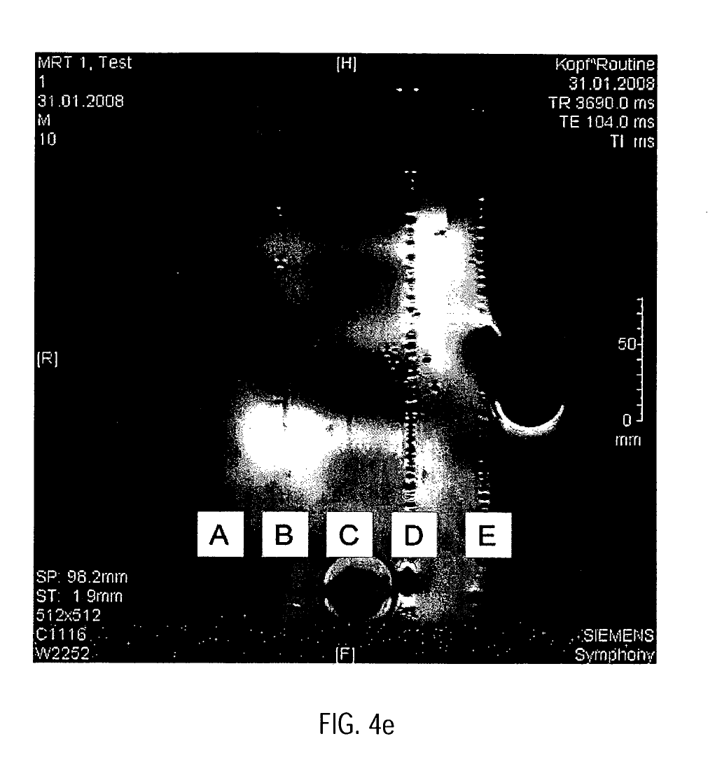

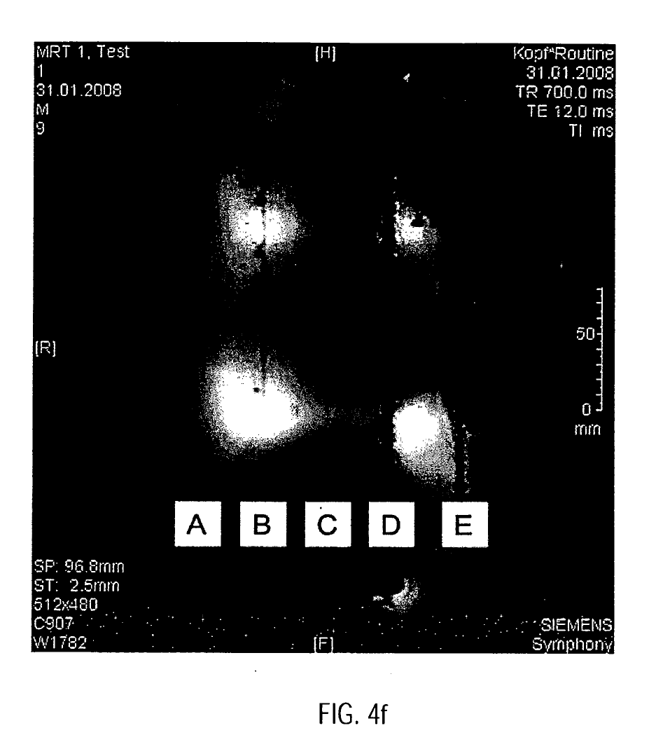

FIGS. 4a to 4f show images which have been created by the test equipment by means of MRT or computer tomography,

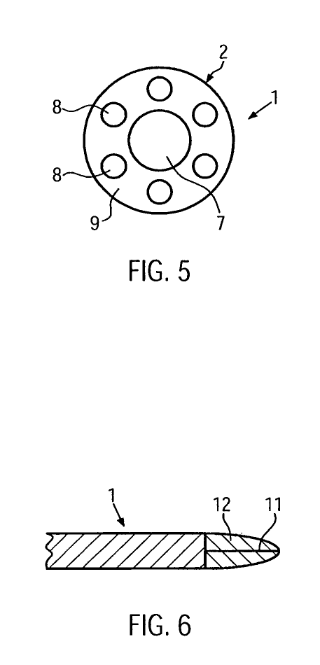

FIG. 5 shows a guiding wire according to a further embodiment of the invention in cross-section,

FIG. 6 shows a guiding wire according to a further embodiment of the invention in a longitudinal section, and

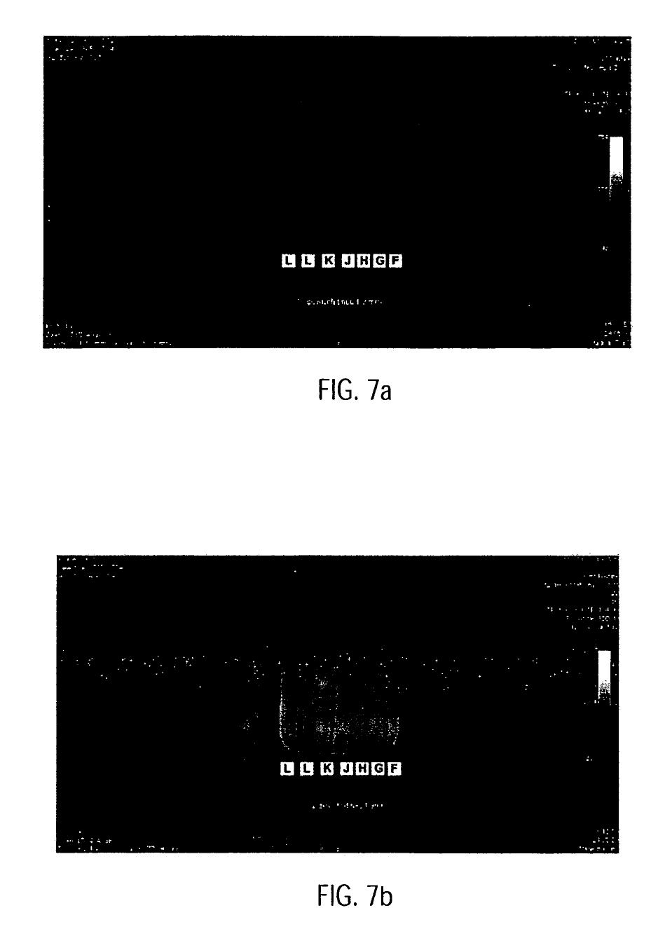

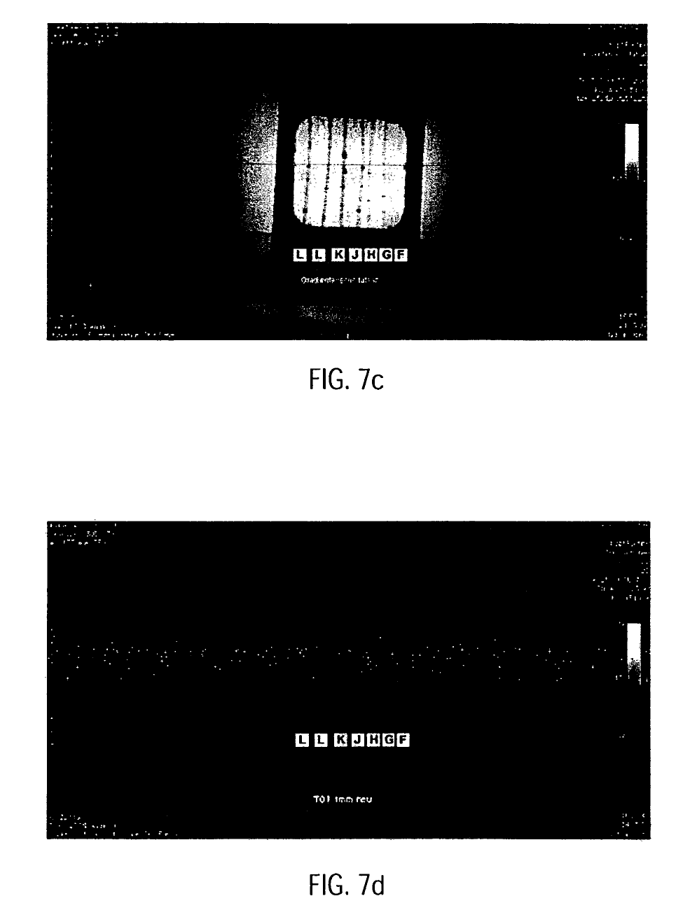

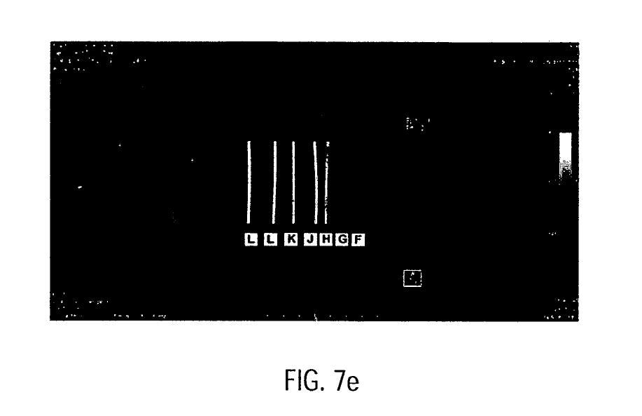

FIGS. 7a to 7e show images which have been created by further test equipment by means of MRT or computer tomography.

The invention will be exemplified in the following on the basis of a guiding wire 1 for a catheter. The guiding wire 1 is made from a material which does not create any MRT artifacts. A material of this kind is, for example, a ceramic or plastic material such as PEEK, PEBAX, PE, PP, PU, silicone, polylactic acid polymers, aromatic polyamides or memory plastic materials. The plastic material is in particular reinforced with fibers. Apart from the above-mentioned plastic materials, epoxy resin can also be used as a matrix material. The fibers are glass fibers or ceramic fibers or Kevlar® fibers, Dacron, plant-based fibers (e.g. silk, sisal, hemp etc.). Materials which do not create any MRT artifacts must be free from electrically conductive sections. The electrically conductive sections should have a length of not more than 15 cm, in particular not more than 10 cm or 5 cm. This is why it is possible to use electrically conductive fibers such as coal-based or carbon fibers, or electrically conductive wires provided that the sections are electrically insulated from one another to a sufficient extent. They must not be formed from a ferromagnetic, paramagnetic, ferrimagnetic or anti-ferromagnetic material.

The guiding wire is an elongated body with a circular cross-section and a diameter of usually not more than 2 mm (e.g. 0.7 mm). On its surface 2, active MR markers 3 are immobilized on the guiding wire.

Active MR markers are markers which interact with a proton-containing medium such as water or fat molecules in such a way that they bring about a quicker relaxation of the protons adjoining the MR marker after their induced orientation by an applied magnetic field. Such MR markers comprise, for instance, an element or a combination of elements or a compound of an element from the group consisting of gadolinium, cerium, praseo-dymium, neodymium, promethium, samarium, europium, terbium, dysprosium, holmium, erbium, thulium, ytterbium, lutetium. These elements are preferably immobilized by means of a complex, in particular by means of a chelate complex. They can also be present as salts or in alloys.

Typical chelating agents are EDTA (ethylenediaminetetraacetic acid), DTPA (diethylenetriaminepentaacetic acid) and DOTA (1,4,7,10-tetrazacyclododecane-tetraacetic acid).

Basically, chemical macromolecules (inter alia polylysines, dendrimers) or biological macromolecules (proteins, sugars, inter alia dextran) are suitable as complexes.

In the present exemplary embodiment, the MR markers are gadolinium(III) chelate complexes, the chelate complexes being bound to the surface 2 of the guiding wire 1 by means of a covalent bond. It is preferred that spacer molecules are provided between the chelate complexes and the surface 2 so that the MR markers are arranged so as to be spaced from the surface 2. Polyethylene glycol is suited for being used as a spacer molecule, for instance.

The covalent bond between the chelates, spacers and the instrument body formed from a polymer can be realized through amino, quaternary ammonium, hydroxyl, carboxyl, sulfhydryl, sulfate, sulfonium, thiol groups, reactive nitrogen groups, etc. (in each case for chelating agents and polymers).

The guiding wire 1 is used for inserting catheters into blood vessels. During inserting the guiding wire 1 in the blood vessel, the surface 2 of the guiding wire 1 comes into contact with blood. Blood flows over and around the MR markers 3 which are arranged so as to be spaced from the surface 2 so that water molecules are attached to the majority of the MR markers 3. The MR markers interact with the water molecules such that their relaxation time is reduced. In an MRT examination, these water molecules produce a high-contrast signal. This is why the guiding wire becomes clearly visible in the image created by MRT. The active MR markers 3 immobilized on the surface 2 of the guiding wire 1 ensure a uniform contrast in all known sequences (for instance T1-weighted, T2-weighted, gradient echo sequence etc.). With conventional medical instruments provided with passive MR markers (e.g. WO 2007/000148 A2) it is also possible to readily detect these markers by means of MRT, but the passive MR markers bring about a disturbance of the field lines which are pronounced to differing extents at different sequences; this often has the effect that with certain sequences the image is disturbed to such a large or small extent that it can not be used for the medical examination. This is why medical instruments provided with passive MR markers cannot be used with all sequences, or the concentration of the passive MR markers is so low or so high that they are not visible any more with certain sequences and result in excessively strong signals overlaying the surrounding structures, respectively.

Medical instruments, provided with active MR markers on their surface like the guiding wire described above, have a considerably more flexible field of application with respect to the sequences compared to instruments with passive MR markers due to the other underlying physical effect and are also uniformly visible in MRT examinations with different sequences.

FIG. 2 shows a second exemplary embodiment of an instrument according to the invention, which again is a guiding wire 1 comprising a surface 2. The body of the guiding wire is designed like the body of the guiding wire according to the first exemplary embodiment. The surface 2 is provided with a coating 4 capable of swelling. Such coatings which are able to swell are formed from polyvinylpyrrolidone (PVP), for instance. Such coatings with swelling ability are available from BASF AG inter alia under the trade name of Colidone or Collidone.

Active MR markers are embedded in the coating with swelling ability. To give an example, a gadolinium(III) chelate complex is used as an MR marker.

When immersed in an aqueous or fatty environment, the coating 4 with swelling ability absorbs water molecules or fat molecules so that the water or fat molecules attach to the active MR markers. The MR markers interact with the protons contained in water and fat molecules so that their relaxation time is reduced and they are visible in an MRT examination.

This embodiment of the guiding wire can also be detected in MRT by means of any sequences. This is why this guiding wire has a very flexible range of use with respect to MRT.

As a rule, the active MR markers are toxic in elementary or free form. When the active markers are bound in complexes, however, they are usually well tolerated by the human and animal bodies. The higher the binding constant in the chelate complex, the lower the dissociation of the MR marker from the complexing agent and hence the risk of elementary MR markers migrating freely into the body fluid. With the invention, the active MR markers are immobilized on the respective medical instrument so that after the examination they are removed from the human or animal body together with the instrument. Therefore, there is a minimum danger in terms of a toxic effect.

The invention has been explained above on the basis of two guiding wires. However, the invention is not limited to guiding wires. Within the scope of the invention, any instruments which can be inserted in human or animal bodies can be realized according to the invention by immobilizing active MR markers in the surface area of the instrument body in such a manner that they are able to interact with the protons in the body medium. Such instruments are, for instance, catheters, stents or implants. The instrument body is preferably formed from a material which does not create any MRT artifacts or only small ones so that the contrast is primarily caused by the active MR markers arranged in the surface area. Materials of this kind are preferably plastic materials, in particular glass-fiber reinforced plastics. They can also be ceramic materials and composite materials from ceramics and plastics.

According to a further aspect of the present invention, the medical instruments are provided with both MR and X-ray markers. It is preferred that active MR markers are used as MR markers in the way explained above. It is also possible, however, to use passive MR markers. Passive MR markers are paramagnetic, ferromagnetic, ferrimagnetic and anti-ferromagnetic metals, metal alloys and metallic compounds. They are preferably embedded in a plastic matrix in the form of particles. The passive MR markers are preferably the following metals or metallic compounds: Cobalt (Co), nickel (Ni), molyb-denum (Mo), zirconium (Zr), titanium (Ti), manganese (Mn), rubidium (Rb), aluminum (Al), palladium (Pd), platinum (Pt), chromium (Cr) or chromium dioxide (CrO₂), and in particular iron (Fe) and iron oxide (FeO, Fe₂O₃, Fe₃O₄). The concentration of the passive MR markers is to be selected such that they are visible with the desired sequences, give a good reproduction of the medical instrument in at least one MR sequence, but do not superpose or impair the imaging of the surrounding body tissue in this process. The active MR markers arranged on the surface are preferred, however, as they can be used in a much more flexible way.

For the X-ray markers, however, the following metals or other elements are used: Barium (Ba), tungsten (W), tantalum (Ta), osmium (Os), praseodymium (Pr), platinum (Pt), gold (Au) and lead (Pb). These elements can be used as X-ray markers in elementary form or also in compounds such as barium sulfate.

Usually, the X-ray markers hardly have an influence on the imaging in an MRT process. In X-ray examinations, for instance in computer tomography or screenings, however, they can be easily detected by means of X-rays.

Some markers can be generally used as both X-ray and passive MR markers, where the imaging function depends on the concentration in each case. As will be explained in more detail below, iron produces image signals in both MRT and X-ray examination. However, the iron concentrations required for the X-ray examination are so high that the image will be disturbed in MRT. Markers which can be used as both X-ray and MR markers are used in such a concentration that they do not disturb either the MRT or the X-ray examination. As a rule, the concentrations of these markers are adjusted such that they only produce an image signal in magnetic resonance imaging and are hardly visible during the X-ray examination. The situation is a similar one if platinum is used but here the difference in the effect is not so marked between the two imaging methods.

The X-ray markers are formed from particles which are embedded in a rod-type body. The rod-type body in turn is part of the medical instrument which may comprise several of these rod-type bodies which can be provided with the same or also with different markers, including passive MR markers. Such a rod-type body is preferably designed as described in WO 2007/000148 A2. Concerning this matter, reference is made to this document.

The rod-type body is formed from a matrix material enclosing non-metallic filaments and the particles of the respective marker. The matrix material is preferably a plastic material such as epoxy resin, PEEK, PEBAX, PE, PP, PU, silicone, polylactic acid polymers. The filaments are glass fibers, ceramic fibers, Dacron, Kevlar® or plant-based fibers (e.g. silk, sisal, hemp etc.), for instance.

The rod-type body is designed so as to have a poor electrical conductivity. Basically, the particles of the markers can have a good electrical conductivity (e.g. iron or platinum particles). However, they are to be provided in such a concentration that they are insulated from one another by the matrix material and at least do not form an electrical conductor which has a length of more than 15 cm and preferably of not more than 10 cm or 5 cm.

The use of such rod-type bodies which normally have a diameter of 0.1 to 0.7 mm and preferably of 0.1 to 0.3 mm, allows the simple manufacture of medical instruments; such medical instrument can be realized in a simple way with different markers by forming it from rod-type bodies provided with different doping agents. The rod-type bodies can be embedded in a further, primary matrix material for forming the medical instrument. They can also be braided to form a medical instrument.

A medical instrument comprising at least one X-ray marker and at least one MR marker can thus be used for both X-ray and MRT examinations and is clearly visible in each case without any disturbance of the imaging process caused by one of the two markers.

FIG. 3 shows test equipment for testing different markers in different imaging methods. The test equipment comprises five test rods 5 arranged on a plastic plate 6. The test rods are each formed from a two-component epoxy resin. One of the test rods 5/1 consists exclusively of the epoxy resin. Two of the test rods, 5/2 and 5/3, are doped with tungsten powder, and two further test rods 5/4, 5/5 are doped with an iron powder. The iron powder is sold by the Roth company under the trade name of Eisenrothipuran under number 3718.1. It has a purity of at least 99.5%. The grain size is in the range of 4 to 6 μm. The tungsten powder is tungsten fine powder 99+ from the Merck KGaA company, marketed under number 1.12406.0100. It has a purity of at least 99.0%. The grain size is smaller than 20 μm. The tungsten powder is paramagnetic. The test rod 5/2 comprises tungsten powder in an amount of 10% by weight. The test rod 5/3 comprises tungsten powder in an amount of 1% by weight. The test rod 5/4 comprises iron powder in an amount of 10% by weight. The test rod 5/5 comprises iron powder in an amount of 1% by weight.

This test equipment was arranged in a tub (filled with water at 37° C.) such that a water layer having a thickness of at least 5 mm was underneath the test equipment and a water layer having a thickness of at least 25 mm was above the test equipment.

This test equipment was subjected to an MRT process with a T1-weighted sequence (FIGS. 4a, 4b), a gradient echo EPI sequence (FIG. 4d), a T2-weighted sequence (FIG. 4e) and a gradient echo sequence (FIG. 4f). Further, the test equipment was subjected to an X-ray examination (CT) (FIG. 4c).

The Figures clearly show that the iron particles, even with comparably low concentrations, are the reason for substantial artifacts in an MRT process, which artifacts have such a disturbing impact on the image in the vicinity of the iron-containing area that it is useless for analysis. This is true in particular for the MRT examination by means of the gradient echo sequence (FIG. 4f).

Tungsten, however, having an atomic number which is much higher than that of iron, can hardly be seen in the MRT examinations as the test rods 5/2 and 5/3 do not produce a higher contrast than the test rod 5/1 which is not doped at all. The test rod 5/2 with an amount of 10% by weight of tungsten powder can be seen very well in the X-ray examination (FIG. 4c). Even the test rod 5/3 which is provided with a very low-rate tungsten doping can still be seen in the X-ray examination.

Basically, it can be said that the elements of the X-ray markers generally have a higher atomic number than the elements of the MR markers, with an overlapping area existing, too. With the exception of platinum (atomic number 78), the preferred passive MR markers have an atomic number of not higher than 46 (palladium). The preferred X-ray markers, however, have an atomic number of at least 56 (barium).

This results in the realization of a medical instrument which can be seen in both MRT and CT and does not induce any disturbances in the image.

FIG. 5 shows a further example of the medical instrument according to the invention which is a guiding wire 1. This guiding wire 1 comprises seven rod-type bodies 7, 8. A central rod-type body 7 is arranged in the center of the guiding wire 1. Six radial rod-type bodies 8 are arranged around the central rod-type body 7 so as to be equally spaced from each other. All rod-type bodies 7, 8 are embedded in a sheathing matrix 9. The surface of the sheathing matrix 9 defines the surface of the guiding wire 1.

As explained above, the rod-type bodies 7, 8 are formed from a matrix material containing non-metallic filaments. The above explanation of the rod-type bodies also applies to the rod-type bodies 7, 8 unless otherwise stated below.

The central rod-type body 7 has a larger diameter than the radial rod-type bodies 8. This results in the central rod-type body 7 having a higher stiffness than the radial rod-type bodies 8. As the central rod-type body 7 is arranged in the center of the guiding wire 1, its higher stiffness has a smaller effect on the flexural rigidity of the whole medical instrument than the radial rod-type bodies 8 as it is arranged on the bending line of the medical instrument. The radial rod-type bodies 8 have a higher flexibility and this is why they do not affect the flexural rigidity of the medical instrument too much. Therefore, a medical instrument is obtained which has a suitable flexibility.

The embodiment illustrated in FIG. 5 is very advantageous as it results in a very thin guiding wire with high strength and flexibility, and due to the radial arrangement of the radial rod-type bodies 8 the guiding wire 1 has a high torsional stiffness.

Further, the strength and flexibility of the medical instrument can be changed by a different number of the rod-type bodies and also by a modified arrangement, for instance without the central rod-type body. The flexibility of the guiding wire is an essential feature and to be individually adapted to different applications. The flexibility of the guiding wire can be varied by varying the diameter of the central rod-type body and/or of the radial rod-type bodies as well as by changing the composition of the sheathing matrix. In order that the medical instrument has the desired strength and flexibility, it is useful that all rod-type bodies are fully enclosed by the sheathing matrix.

The radial rod-type bodies 8 may extend parallel to the central rod-type body 7. However, they can also be arranged in a spiral arrangement around the central rod-type body 7.

The central rod-type body has a diameter of 0.1 to 0.4 mm, preferably from approximately 0.2 to 0.3 mm. The central rod-type body is doped with tungsten nano particles (particle size approximately 40 to 50 nm), for example.

The amount of the tungsten particles in relation to the matrix material of the rod-type body is 50% by weight. In the present embodiment, an epoxy resin adds the remaining 50% by weight. The rod-type body additionally comprises glass fibers.

It has turned out that the tungsten nano particles during manufacturing the rod-type body have had an advantageous influence on the flowability of the epoxy resin. The undoped rod-type bodies are extruded with the addition of aerosils in order to improve the flowability. In case tungsten particles are used, adding such aerosils to the epoxy resin is not necessary. It has turned out that the smaller the particles, the better the viscosity of the epoxy resin.

With a high amount of tungsten particles, these act as both X-ray and MR markers. The weight proportion of the tungsten particles in relation to the matrix material should be at least 1:2 to 2:1. The higher the amount of the tungsten particles, the better their effect as MR markers. This effect as MR markers also depends on the size of the rod-type body and hence on the absolute amount of the tungsten particles and the particle size of the tungsten particles. Tungsten particles with a size from a few pm to approximately 20 μm are hardly suited as MR markers as explained above on the basis of FIGS. 4a, 4b and 4d to 4f. The smaller the tungsten particles, the higher their effect as MR markers. It has tuned out that the weight proportion of the tungsten particles in relation to the matrix material can be adjusted up to a range of 2:1 to 3:1.

The radial rod-type bodies 8 have a diameter from 0.10 to 0.25 mm, preferably from 0.15 to 0.20 mm. Only one of the radial rod-type bodies 8 is doped with Fe₃O₄ particles in the present embodiment. The particles have a particle size of approximately 40 to 50 nm. The particles should have a size of not more than 100 nm, preferably not more than 60 nm. In the doped radial rod-type body 8, one part by weight of Fe₃O₄ particles accounts for approximately 10 to 30, preferably 20 to 25 parts by weight of the matrix material which preferably is epoxy resin again. The Fe₃O₄ particles are passive MR markers.

Within the scope of the invention it is also possible, of course, to dope the rod-type bodies with other passive markers, other concentrations and other particles sizes. It is also possible to provide more than two rod-type bodies with a marker, preferably with different markers. The number, the arrangements and the diameters of the rod-type bodies can also vary.

It is also possible that several different markers are provided in one rod-type body.

Within the scope of the invention it is also possible to provide this guiding wire on the surface with one of the coatings described above and containing an active MR marker.

The sheathing matrix 9 is a thermoplastic elastomer, preferably polyurethane, in particular Tecoflex™ or Mediprene®.

Mediprene® is a thermoplastic elastomer which is primarily used for medical purposes. Mediprene is offered by VTC Elastoteknik AB, Sweden. Mediprene® is understood to mean Mediprene® TO 34007, a thermoplastic elastomer made from SEBS (styrene-ethylene-butylene-styrene-elastomer).

The medical instrument shown in FIG. 5 is preferably manufactured by co-extruding the rod-type body and the sheathing matrix.

The use of rod-type bodies with different doping agents is not restricted to guiding wires. Rod-type bodies with different doping agents can also be used with other medical instruments such as catheters, stents or implants.

It is preferred that a guiding wire 1 according to one of the above exemplary embodiments is provided with a flexible tip (FIG. 6). The flexible tip 10 is made from an axial nylon thread 11 and a polyurethane body 12. This flexible tip 10 is produced by coating the nylon thread step by step so that the flexible tip 10 can be formed as a blunt tip. The flexible tip is connected with a front face of the guiding wire 1 by means of a glued connection. It is preferred that the flexible tip 10 is doped with one of the passive doping agents described above and/or coated with an active marker.

The front face of the guiding wire 1 and the corresponding contact surface of the flexible tip 10 are preferably ground so as to be cone-shaped so that the contact area between the guiding wire 1 and the flexible tip 10 is enlarged.

The flexible tip 10 can also be connected with the guiding wire 1 by heating the two contact surfaces. It is also possible to solubilize the flexible tip 10 with a chemical solvent (e.g. in solution grade polyurethane) and connect it with the guiding wire 1 in this way. A suitable solvent is THF, for instance, if polyurethane is used as the material for the flexible tip 10. Instead of polyurethane, epoxy resin, PEEK, PEBAX, PE, PP, silicone, polylactic acid or Mediprene® can also be used as the material for the flexible tip 10. The axial polymer thread can also be formed from other materials, for instance from PEEK, PEBAX, PE, PP, silicone or polylactic acid. The flexible tip can also be realized without an axial thread.

The nylon thread is preferably doped with a marker. It can be doped with a marker which is different from the marker of the remaining material of the flexible tip 10. In case there is no thread, the material for the flexible tip can be doped with a marker.

FIGS. 7a to 7e show further test equipment created by means of MRT or X-ray tomography.

With this test equipment, rod-type bodies, on the one hand, and guiding wires in water, on the other hand, were examined.

The rod-type bodies generally consist of epoxy resin with glass fibers. The following different rod-type bodies were examined:

(F) Diameter 0.17 mm; no doping

(G) Diameter 0.17 mm; doped with Fe₃O₄ nano particles; weight ratio between doping agent and epoxy resin is 1:20

(H) Diameter 0.27 mm; doped with tungsten nano particles; weight ratio of doping agent to epoxy resin 1:1

(J) Diameter 0.27 mm; doped with tungsten nano particles; weight ratio of doping agent to epoxy resin 2:1

The examined guiding wires 1 have basically the structure which is shown in FIG. 5 and has been described on the basis of FIG. 5, with the central rod-type body 7 having a diameter of 0.27 mm and being doped with tungsten nano particles. The radial rod-type bodies 8 have a diameter of 0.17 mm. Five radial rod-type bodies 8 are undoped. One of the radial rod-type bodies 8 is doped with Fe₃O₄ nano particles.

The following guiding wires were examined:

(K) Sheathing matrix made from polyurethane; doping amount of the central rod-type body of tungsten nano particles in a weight ratio of 1:1 in relation to the epoxy resin, a radial rod-type body 8 doped with Fe₃O₄, the weight ratio of doping agent to epoxy resin being 1:20;

(L) Sheathing matrix made from Mediprene®; doping amount of the central rod-type body of tungsten nano particles in a weight ratio of 2:1 in relation to the epoxy resin, a radial rod-type body 8 doped with Fe₃O₄, the weight ratio of doping to epoxy resin being 1:20;

FIG. 7a shows a T1-weighted MRT sequence, FIG. 7b a T2-weighted MRT sequence, FIG. 7c an MRT gradient echo sequence, and FIG. 7d an MRT Angio TOF sequence. FIG. 7e shows a computertomographic illustration of the rod-type bodies and guiding wires.

The undoped rod-type body F can be hardly seen in any of the Figures. Due to the displacement of the water in the test equipment, traces with partially a very low contrast can be seen in the MRT.

The radial rod-type body doped with Fe₃O₄ is visible in the MRT process with differing contrast. In the MRT gradient echo sequence and the MRT Angio TOF sequence, the contrast is high, and in the two T1- and T2-weighted sequences the contrast is low. The rod-type body H doped with tungsten nano particles has shown similar results with MRT, with the contrasts with the two T1- and T2-weighted MRT sequences being better than that of the rod-type body G. Further, the rod-type body H produces an excellent contrast even in computer tomography (X-ray examination).

Such a rod-type body doped with tungsten nano particles (particle size smaller than 100 nm, preferably smaller than 60 nm) represents a separate, independent idea of the invention as the use of such a rod-type body in a medical instrument in itself produces the visualization of the medical instrument both in X-ray and MRT examinations. Using other markers, better contrasts can be achieved in part so that a combination with further markers still makes sense but is not absolutely necessary. Tungsten nano particles also have the advantage that they produce a good contrast in both X-ray and MRT examinations in a predetermined concentration in the rod-type body. Basically, iron particles are also suited for creating a contrast in both X-ray and MR examinations. With iron particles, however, there is the problem that they produce large artifacts with higher concentrations which are the cause of heavy disturbances of the image in a larger surrounding. With low concentrations suitable for MRT, the iron particles are not visible in an X-ray examination.

Further, it is to be seen from FIGS. 7a to 7d that the doped rod-type bodies as well as the guiding wires containing doped rod-type bodies can all be seen clearly in the tested MRT sequences.

### LIST OF REFERENCE NUMERALS

1 Guiding wire
2 Surface
3 MR marker
4 Coating capable of swelling
5 Test rod
6 Plastic plate
7 Central rod-type body
8 Radial rod-type body
9 Sheathing matrix
10 Flexible tip
11 Nylon thread
12 Polyurethane body

1. A medical instrument which can be inserted in a human or animal body,
the medical instrument including an instrument body which comprises at least one rod-type body having poor electrical conductivity and being formed from a matrix material and non-metallic filaments,
characterized in that
the rod-type body is doped with X-ray marker particles and the medical instrument comprises an MR marker.

2. The medical instrument, in particular according to claim 1, which can be inserted in a human or animal body,
the medical instrument including an instrument body which comprises at least one rod-type body having poor electrical conductivity and being formed from a matrix material and non-metallic filaments, the rod-type body, being doped with an X-ray marker and/or MR marker,
characterized in that
the instrument body is provided with an immobilized active MR marker in the surface area.

3. The medical instrument according to claim 1,
characterized in that
the rod-type body has a diameter from 0.1 to 0.7 mm, preferably from 0.1 to 0.3 mm.

4. The medical instrument according to claim 1,
characterized in that
the rod-type body is doped with tungsten particles which are contained in the rod-type body in a ratio of at least 50% by weight in relation to the matrix material of the rod-type body so that the tungsten particles form an MR marker as well as an X-ray marker.

5. The medical instrument according to claim 1,
characterized in that
the medical instrument comprises at least one additional rod-type body which is doped with an additional marker, in particular a passive MR marker.

6. The medical instrument according to claim 1,
characterized in that
the at least one rod-type body is doped with at least two different markers.

7. The medical instrument according to claim 2,
characterized in that
the active MR markers are immobilized in the surface area by means of complexes and the active MR markers comprise an element or a combination of elements or compounds of the elements selected from the group consisting of gadolinium, cerium, praseodymium, neodymium, promethium, samarium, europium, terbium, dysprosium, holmium, erbium, thulium, ytterbium, and lutetium.

8. The medical instrument according to claim 1,
characterized in that
the medial instrument has no elongated, electrically conductive sections and is formed from a non-ferromagnetic material.

9. The medical instrument according to claim 1,
characterized in that
the X-ray markers comprise one or more of the following elements or compounds with one or more of the following elements such as barium (Ba), tungsten (W), tantalum (Ta), osmium (Os), praseodymium (Pr), platinum (Pt), gold (Au), lead (Pb).

10. The medical instrument according to claim 1,
characterized in that
the MR marker is a passive MR marker and formed from a paramagnetic, ferromagnetic, ferrimagnetic and anti-ferromagnetic metal, metal alloy or metallic compound.

11. The medical instrument according to claim 10,
characterized in that
the passive MR marker comprises a metal or metal alloy or metallic compound comprising cobalt (Co), nickel (Ni), molybdenum (Mo), zirconium (Zr), titanium (Ti), manganese (Mn), rubidium (Rb), aluminum (Al), palladium (Pd), platinum (Pt), chromium (Cr) or iron (Fe).

12. The medical instrument according to any of claims 1 to 11,
characterized in that
it comprises several MR markers which are optimized for different sequences such as T1-weighted, T2-weighted or gradient echo.

13. The medical instrument according to claim 1,
characterized in that
the instrument body is a body formed by co-extruding a sheathing matrix and one or more rod-type bodies, the sheathing matrix being formed from a thermoplastic elastomer.

14. The medical instrument according to claim 1,
characterized in that
the medical instrument is a catheter, a guiding wire for a catheter, a stent or an implant.

15. A method of detecting a medical instrument in a human or animal body, wherein
a medical instrument according to claim 1 is inserted in the human or animal body, and said medical instrument is detected by means of magnetic resonance tomography or X-ray tomography.

16. The medical instrument according to claim 7, characterized in that the complexes comprise chelate complexes.

17. The medical instrument according to claim 7, characterized in that the active complexes are covalently bound to the instrument body or embedded in a coating capable of swelling and formed on the surface of said instrument body.
